(19) United States
(12) Patent Application Publication
(10) Pub. No.: US 2016/0228377 A1
(43) Pub. Date: Aug. 11, 2016

(54) METHOD FOR THE TREATMENT OF DIABETES MELLITUS

(71) Applicant: BETA-CELL NV, Zellik (BE)

(72) Inventors: Myriam Lea Willy Bomans, Nieuwenrode (BE); Mike De Leeuw, Maastricht (NL)

(73) Assignee: Beta-Cell NV, Zellik (BE)

(21) Appl. No.: 15/022,887

(22) PCT Filed: Sep. 19, 2014
(86) PCT No.: PCT/EP2014/070028

§ 371 Date: Mar. 17, 2016

(30) Foreign Application Priority Data
Sep. 19, 2013 (EP) 13185242.8

(51) Int. Cl. A61K 9/48; A61K 9/00; A61L 27/20; A61L 27/58; A61L 27/38; A61K 35/39; A61L 27/54

(52) U.S. Cl. CPC A61K 9/4816; A61K 35/39; A61K 9/0019; A61L 27/54; A61L 27/58; A61L 27/3804; A61L 27/20; A61K 2035/128

(57) ABSTRACT

This disclosure is in the field of medical therapy; in particular, it concerns the use of encapsulated cells in cell therapy. More in particular, this disclosure relates to the second medical use of a composition comprising encapsulated cells. Even more in particular, the disclosure relates to the use of a foreign body, suitable for implantation into a subject at a predefined location, wherein the foreign body comprises cells encapsulated in high-M alginate for inducing or stimulating angiogenesis.

### CROSS-REFERENCE TO RELATED APPLICATIONS

This application is a national phase entry under 35 U.S.C. § 371 of International Patent Application PCT/EP2014/070028, filed Sep. 19, 2014, designating the United States of America and published in English as International Patent Publication WO 2015/040176 A1 on Mar. 26, 2015, which claims the benefit under Article 8 of the Patent Cooperation Treaty to European Patent Application Serial No. 13185242.8, filed Sep. 19, 2013.

### TECHNICAL FIELD

This disclosure is in the field of medical therapy; in particular, it concerns the use of encapsulated cells in cell therapy.

### BACKGROUND

Nowadays, cell-based therapy is rarely in clinical practice because of the limited availability of appropriate cells. To apply cells therapeutically, they must not cause any immune response and it is because of that reason that mainly autologous cells have been used up to now. (Freimark et al., Transfus. Med. Hemother. 2010, 37:66-73]. The amount of vital cells in patients is limited, however, and under certain circumstances, such as in highly degenerated tissues, no vital cells are left. Moreover, the extraction of these cells is connected with additional surgery. In addition, the expansion in vitro is difficult.

Other approaches avoid these problems by using allo- or even xenogenic cells. These cells are more stable concerning their therapeutic behavior and can be produced in stock. This approach, however, suffers from the disadvantage that the immune system of the host may recognize the allo- or xenogenic cells as foreign. To prevent an immune response caused by these cells, cell encapsulation has been suggested and successfully employed. Certain studies showed that encapsulated allo- and xenogenic cells achieve promising results in treatment of several diseases.

The above studies suffer from the disadvantage that encapsulated cells have a short life time, which is in a large number of studies attributed to oxygen deprivation and shortage of nutrient supply to the encapsulated cells.

In a recent paper (Writer et al., Tissue Engineering 16:1503-1513 (2010)), it was described that adult porcine pancreatic islets that were encapsulated in macroscopic structures, composed of an alginate with a high mannuronic acid content and with a high viscosity, were able to survive for a period of 12 weeks and were able to down-regulate blood glucose levels in diabetic rats for 60 days when implanted subcutaneously.

Notwithstanding these promising results, it remains to be desired to have means and methods that are less invasive and longer lasting.

### BRIEF SUMMARY

This disclosure relates to the second medical use of a composition comprising encapsulated cells. More in particular, the disclosure relates to the use of a foreign body for implantation into a subject at a predefined location, wherein the foreign body comprises fetal, newborn or perinatal pancreatic cells encapsulated in alginate with a high mannuronic acid content (high-M alginate or HM alginate). Such compositions were found to induce or stimulate angiogenesis.

Phrased in more general terms, the disclosure relates to a method of treating a subject suffering from a medical condition or disease characterized by a deficit or shortage of a particular compound, wherein encapsulated cells are provided that produce that compound or induce that compound to be produced by the subject and wherein the encapsulated cells are transplanted into the subject. In a method according to the disclosure, the cells remain functional for a prolonged amount of time.

In a more particular embodiment, the disclosure provides a composition for use in the treatment of a subject with diabetes mellitus, wherein the treatment comprises implanting of the composition into the body of the subject at a predefined location and wherein the composition comprises aggregates of fetal, newborn or perinatal pancreatic cells encapsulated in alginate particles with a mannuronic acid content of at least 50%, and a viscosity of 100 mPa·s or less, wherein the aggregates are smaller than 100 micrometers.

This disclosure concerns the use of encapsulated cells in cell therapy. The term “cell therapy” is used herein as to mean a process of introducing new cells in a body such as a human body, in order to treat a disease or to restore the function of a tissue.

No single cell or universal donor can be used for the treatment of all diseases so that consequently the source and the desired function of the cell will dictate which cell type is most useful for each disease (F. H. Gage, “Cell Therapy,” Nature 1998; 392 (6679 suppl):18-24). There are several forms of cell therapy: i) the transplantation of autologous or allogenic stem cells, ii) the transplantation of mature, functional cells, iii) the transplantation of modified human cells that produce a needed substance, iv) the transplantation of trans-differentiated cells, and v) xenotransplantation.

Although autologous cells have the advantage of causing no immune response and, therefore, are preferred for cell therapy, the retrieval of appropriate cells in sufficient amounts is difficult. Several diseases are congenital so that potential genetic dispositions causing the disease to be treated are still present in autologous cells. Furthermore, additional surgery is needed. For these reasons, allogenic or even xenogenic cells are attractive cell sources for regenerative medicine. To protect these cells from the immune response and to attain cell survival, encapsulation of such cells is a feasible way.

“Cell encapsulation” is an art-recognized term and is used herein to mean the immobilization of cells within a semi-permeable membrane that allows the diffusion of small molecules (therapeutic proteins, nutrients, oxygen, etc.) but protects the cell from the host's immune system and also from mechanical stress (P. J. Morris, “Immunoprotection of therapeutic cell, transplants by encapsulation,” Trends Biotechnol. 1996; 14(5):163-167; and B. Rihova, “Immunocompatibility and biocompatibility of cell delivery systems,” Adv. Drug Deliv. Rev. 2000; 42 (1-2):65-80).

There are many biomaterials such as agar, alginate, carrageenan, cellulose and its derivatives, chitosan, collagen, gelatin, epoxy resin, photo cross-linkable resins, polyacrylamide, polyester, polystyrene and polyurethane, polyethylene glycol (PEG) and other polymers that are used for encapsulation. Existing materials are designed and modified to achieve ideal biocompatibility, degradation and physical properties depending on the field of application (R. H. Li, “Materials for immuno-isolated cell transplantation,” Adv. Drug Deliv. Rev. 1998; 33 (1-2):87-109). One of the preferred materials for cell encapsulation is alginate, which forms a three-dimensional structure after reacting with multivalent cations.

Similar to the available biomaterials, the formation methods are multifaceted as well. One of the preferred methods is the formation of a core capsule covered by an outer layer. This technology has been described extensively in H. Uludag, P. De Vos, and P. A. Tresco: “Technology of mammalian cell encapsulation,” Advanced Drug Delivery Rev. 2000; 42(1-2):29-64; M. S. Shoichet, R. H. Li, M. L. White, and S. R. Winn: “Stability of hydrogels used in cell encapsulation: An in vitro comparison of alginate and agarose,” Biotechnol. Bioeng. 1996; 50(4):374-381; H. Zimmermann, S. G. Shirley, and U. Zimmermann: “Alginate-based encapsulation of cells: past, present, and future,” Curr. Diab. Rep. 2007; 7 (4):314-320; and J. M. Rabanel, X. Banquy, H. Zouaoui, M. Mokhtar, and P. Hildgen: “Progress technology in microencapsulation methods for cell therapy,” Biotechnol. Prog. 2009; 25(4):946-963, the teachings of which are incorporated by reference herein.

Many diseases, particularly chronic diseases, are based on a dysfunction of certain cell types. Cellular processes are very complex, with many regulation and signaling pathways that cannot be imitated in vitro that easily. Therefore, it is very difficult to develop drugs or therapies for the treatment of such diseases based on in vitro studies because the results of these studies are often not able to project a drug effect in vivo. More successful is the implementation of cells that produce a therapeutic protein or restore the tissue function because this corresponds more to the natural behavior and can minimize unintentional side effects.

Compared to alternative therapies, the advantages of cell encapsulation are the use of allogenic (non-human) cells as an alternative source to the limited supply of donor tissue, the avoidance of permanent immunosuppression, and, if desired, the delivery of a therapeutic product over long time periods. In addition, genetically modified cells can be induced to produce any protein in vivo without changes in the patient's genome. In comparison to the encapsulation of a therapeutic protein alone, the immobilization of cells allows a continuous and controlled release of a de novo synthesized protein with a constant rate, giving rise to more physiological conditions. Moreover, in the case of capsule damage, the fast release of high protein concentrations causing toxicity can be avoided. Due to these reasons, cell therapy with encapsulated cells seems to be a promising approach for many clinical applications.

Encapsulated primary cells are also known as “bio-organs” or “bio-hybrids.” Most common is the implementation of encapsulated islets of Langerhans mimicking the pancreas for the treatment of diabetes. For this disorder, promising results from animal studies were obtained in allo- and xenotransplantation approaches (P. Gianello and D. Dufrane: “Correction of a diabetes mellitus type 1 on primate with encapsulated islet of pig pancreatic transplant,” Bull. Mem. Acad. R. Med. Belg. 2007; 162(10-12):439-450; C. G. Thanos and R. B. Elliott: “Encapsulated porcine islet transplantation: an evolving therapy for the treatment of type I diabetes,” Expert Opin. Biol. Ther. 2009; 9(1):29-44; S. P. Black, I. Constantinidis, H. Cui, C. Tucker-Burden, C. J. Weber, and S. A. Safley: “Immune responses to an encapsulated allogeneic islet beta-cell line in diabetic NOD mice,” Biochem. Biophys. Res. Commun. 2006; 340(1):236-243; and J. J. Altman, D. Houlbert, A. Chollier, A. Leduc, P. McMillan, and P. M. Galletti: “Encapsulated human islet transplants in diabetic rats,” Trans. Am. Soc. Artif. Intern. Organs 1984; 30:382-386).

Moreover, initial pilot clinical trials have been made that came to the conclusion that to some extent, the function of the pancreas could be restored although the medication with insulin could not completely be discontinued (R. B. Elliott, L. Escobar, P. L. Tan, M. Muzina, S. Zwain, and C. Buchanan: “Live encapsulated porcine islets from a type 1 diabetic patient 9.5 yr after xenotransplantation,” Xenotransplantation 2007; 14(2):157-161; A. J. Scheen: “Clinical study of the month. Prolonged insulin independence after transplantation of islets of Langerhans in a patient with type 1 diabetes: achievement of a dream?” Rev. Med. Liege 2000; 55(8):803-805; and R. Calafiore, G. Basta, G. Luca, A. Lemmi, M. P. Montanucci, G. Calabrese, L. Racanicchi, F. Mancuso, and P. Brunetti: “Microencapsulated pancreatic islet allografts into non-immunosuppressed patients with type 1 diabetes: first two cases,” Diabetes Care 2006; 29(1):137-138).

Primary cells are also used in the treatment of neurodegenerative disease such as Huntington's disease. This disease is caused by a mutation of the protein Huntington, which results in the damage of specific areas of the brain. Choroid plexi (CPs) are areas in the brain that produce the cerebrospinal fluid (CSF) and act as a filtration system, removing metabolic waste, foreign substances and excess neurotransmitters from the CSF. In this way, the CP helps to maintain an extracellular environment required for optimal brain function (D. F. Emerich, A. V. Vasconcellos, R. B. Elliott, S. J. Skinner, and C. V. Borlongan: “The choroid plexus: function, pathology and therapeutic potential of its transplantation,” Expert Opin. Biol. Ther. 2004; 4(8):1191-1201).

In several studies concerning Huntington's disease, CP was encapsulated and transplanted in the brain of laboratory animals. In a primate model of Huntington's disease, encapsulated CP delivers neurotrophic growth factors that prevent neurons from degeneration. Furthermore, the secretion of neuroactive substances by encapsulated bovine chromaffin cells gave promising results in the rat model in the treatment of chronic neuropathic pain, another neural disorder (Y. Jeon, K. Kwak, S. Kim, Y. Kim, J. Lim, and W. Back: “Intrathecal implants of microencapsulated xenogenic chromaffin cells provide a long-term source of analgesic substances,” Transplant Proc. 2006; 38(9):3061-3065). Genetically engineered mature cells have also been used for cell therapy. The number of primary cells is limited, therefore, other cell sources are needed. One alternative is the genetic engineering of mature cells to deliver the desired therapeutic protein. Which cell type will be used depends on the application, as well as on processing, storage, availability and costs. For some applications such as cerebral and cardiovascular disorders that are difficult to treat with common medication, encapsulated genetically improved cells could be more suited for therapy as common medicaments. Genetically engineered fibroblasts (recombinant glial cell line-derived neurotrophic factor (GDNF) production) were encapsulated and investigated concerning their ability in the treatment of Parkinson's disease. In a rat model, GDNF delivery improved the restoration of the nerve function (T. Yasuhara and I. Date: “Intracerebral transplantation of genetically engineered cells for Parkinson's disease: toward clinical application,” Cell Transplant 2007; 16(2):125-132; A. Sajadi, J. C. Bensadoun, B. L. Schneider, C. LoBianco, and P. Aebischer: “Transient striatal delivery of GDNF via encapsulated cells leads to sustained behavioral improvement in a bilateral model of Parkinson disease,” Neurobiol. Dis. 2006; 22(1):119-129).

An encapsulated cell-based system consisting of engineered murine myoblasts was developed to deliver arylsulfatase A to the CNS of metachromatic leukodystrophy patients (A. Consiglio, S. Martino, D. Dolcetta, G. Cusella, M. Conese, S. Marchesini, G. Benaglia, L. Wrabetz, A. Orlacchio, N. Deglon, P. Aebischer, G. M. Severini, and C. Bordignon: “Metabolic correction in oligodendrocytes derived from metachromatic leukodystrophy mouse model by using encapsulated recombinant myoblasts,” J. Neurol. Sci. 2007; 255(1-2):7-16).

A commonly encountered problem with implants comprising cells is that the cells become deprived of oxygen and nutrients and, therefore, cannot develop or metabolize in an optimal way. Therefore, it has been tried to have foreign grafts produce substances that promote the growth of blood vessels, such as vascular endothelial growth factor.

Vascular endothelial growth factor (VEGF) is a signal protein produced by cells that stimulates vasculogenesis and angiogenesis. It is part of the system that restores the oxygen supply to tissues when blood circulation is inadequate. Serum concentration of VEGF is high in bronchial asthma and low in diabetes mellitus. VEGF's normal function is to create new blood vessels during embryonic development, new blood vessels after injury, muscle following exercise, and new vessels (collateral circulation) to bypass blocked vessels.

When VEGF is overexpressed, it can also contribute to disease. Solid cancers cannot grow beyond a limited size without an adequate blood supply; cancers that can express VEGF are able to grow and metastasize. Overexpression of VEGF can cause vascular disease in the retina of the eye and other parts of the body. Drugs such as bevacizumab can inhibit VEGF and control or slow those diseases.

VEGF is a sub-family of growth factors; to be specific, the platelet-derived growth factor family of cystine-knot growth factors. They are important signaling proteins involved in both vasculogenesis (the de novo formation of the embryonic circulatory system) and angiogenesis (the growth of blood vessels from pre-existing vasculature).

The most important member is VEGF-A. Other members are Placenta growth factor (PGF), VEGF-B, VEGF-C and VEGF-D. The latter ones were discovered later than VEGF-A and, before their discovery, VEGF-A was just called “VEGF.”

Activity of VEGF-A has been studied mostly on cells of the vascular endothelium, although it does have effects on a number of other cell types (e.g., stimulation monocyte/macrophage migration, neurons, cancer cells, and kidney epithelial cells).

In vitro, VEGF-A has been shown to stimulate endothelial cell mitogenesis and cell migration. VEGF-A is also a vasodilator and increases microvascular permeability and was originally referred to as vascular permeability factor.

To treat myocarditis, microencapsulated xenogenic Chinese hamster ovary (CHO) cells expressing vascular endothelial growth factor (VEGF) were implanted into rats. The immune response to the encapsulated CHO cells was low. The VEGF-expressing CHO cells significantly increased angiogenesis, resulting in improvement of heart function (H. Zhang, S. J. Zhu, W. Wang, Y. J. Wei, and S. S. Hu: “Transplantation of microencapsulated genetically modified xenogeneic cells augments angiogenesis and improves heart function,” Gene Ther. 2008; 15(1):40-48).

Other approaches using encapsulated, genetically modified cells are the expression of beta-glucuronidase in epithelial cells treating mucopolysaccharidosis type VII (H. Nakama, K. Ohsugi, T. Otsuki, I. Date, M. Kosuga, T. Okuyama, and N. Sakuragawa: “Encapsulation cell therapy for mucopolysaccharidosis type VII using genetically engineered immortalized human amniotic epithelial cells,” Tohoku J. Exp. Med. 2006; 209(1):23-32), the expression of erythropoietin in myoblast cells (A. Murua, M. de Castro, G. Orive, R. M. Hernandez, and J. L. Pedraz: “In vitro characterization and in vivo functionality of erythropoietin-secreting cells immobilized in alginate-poly-L-lysine-alginate microcapsules,” Biomacromolecules 2007; 8(11):3302-3307; and A. Murua, G. Orive, R. M. Hernandez, and J. L. Pedraz: “Xenogeneic transplantation of erythropoietin-secreting cells immobilized in microcapsules using transient immunosuppression,” J. Control Release 2009; 137(3):174-178), or the expression of IL-6 in CHO cells, inhibiting tumor progression (D. M. Moran, L. G. Koniaris, E. M. Jablonski, P. A. Cahill, C. R. Halberstadt, and I. H. McKillop: “Microencapsulation of engineered cells to deliver sustained high circulating levels of interleukin-6 to study hepatocellular carcinoma progression,” Cell Transplant 2006; 15(8-9):785-798).

It was also found that encapsulated VEGF-producing cells could provide neuroprotective and angiogenic effects on focal cerebral ischemia. Therein, recombinant BHK cells producing VEGF were implanted in rat striata. The grafts provided a neuroprotective and angiogenic effect after an experimentally induced infarct (Yano et al., J. Neurosurg. 2005 103:104-114).

Diabetic mice were also treated with rat pancreatic islets encapsulated in an AN69 membrane together with VEGF. Upon transplantation of these grafts in the peritoneum of mice, insulin secretion of the islets was higher and the glycemia levels decreased and remained stable for up to 28 days (Sigrist et al., Cell Transplant 2003 12:627-635).

In contrast, some studies also show that VEGF-producing grafts may induce accumulation of giant cells and eosinophils around the transplantation site, leading to complete graft encapsulation. It is mentioned that the use of growth factors may produce harmful side effects (Heidenhain et al., Artif. Organs 2011 35:E1-10).

From the above, it may be concluded that allo- or xenogenic mature cells are readily available and, therefore, this does not constitute a major problem. However, cell therapy based on encapsulated allo- or xenogenic cells still has some limitations. Up to now, the cells were found to secrete the therapeutic proteins only for a short time, and a host immune response often occurs in spite of cell encapsulation.

In the research leading to this disclosure, the focus has been on the angiogenesis induced by alginate grafts and cells encompassed in alginate grafts, wherein, in particular, the focus has been on VEGF-producing cells. The goal was to achieve an environment for the cells to continue to metabolize and produce compounds for the treatment of a variety of diseases over a long period of time, such as several weeks or months, notably more than 10, 15 or even 20 weeks.

Angiogenesis is the physiological process through which new blood vessels form from pre-existing vessels. This is distinct from vasculogenesis, which is the de novo formation of endothelial cells from mesoderm cell precursors. The first vessels in the developing embryo form through vasculogenesis, after which angiogenesis is responsible for most, if not all, blood vessel growth during development and in disease.

Angiogenesis is a normal and vital process in growth and development, as well as in wound healing and in the formation of granulation tissue. However, it is also a fundamental step in the transition of tumors from a benign state to a malignant one, leading to the use of angiogenesis inhibitors in the treatment of cancer. Stimulation of angiogenesis may be performed by various angiogenic proteins, including several growth factors, such as VEGF.

Insulin is a hormone that brings about effects that reduce blood glucose concentration. Beta cells can respond quickly to spikes in blood glucose concentrations by secreting some of its stored insulin while simultaneously producing more.

When the glucose concentration outside the cell is high, glucose molecules move into the cell by facilitated diffusion, driven by its concentration gradient through the GLUT2 transporter. Since beta cells use glucokinase to catalyze the first step of glycolysis, metabolism only occurs around physiological blood glucose levels and above. Metabolism of the glucose produces ATP, which increases the ATP to ADP ratio.

The ATP-sensitive potassium ion channels close when this ratio rises. This means that potassium ions can no longer diffuse out of the cell. As a result, the potential difference across the membrane becomes more positive (as potassium ions are accumulating inside the cell). This change in potential opens the voltage-gated calcium channels, which allows calcium ions from outside the cell to diffuse in, down their concentration gradient. When the calcium ions enter the cell, they cause vesicles containing insulin to move to and fuse with the cell surface membrane, releasing insulin by exocytosis.

As a model system, porcine perinatal beta cells encapsulated into an alginate matrix were employed. Beta cells are a type of cell in the pancreas located in the islets of Langerhans. They make up 65-80% of the cells in the islets. The primary function of a beta cell is to produce, store and release insulin.

Beta Cells also produce C-peptide as a result of the cleavage of pro-insulin into C-peptide and insulin, which is secreted into the bloodstream in equimolar quantities to insulin. C-peptide helps to prevent neuropathy and other vascular deterioration-related symptoms of diabetes mellitus. As insulin has a much shorter half-life time in serum than C-peptide, a skilled person measures the levels of C-peptide to obtain an estimate for the amount of insulin produced and the viable beta cell mass.

In the model system, beta cells are encapsulated with a specific type and quality of alginate to form alginate particles, also referred to as micro-capsules. Alginate comprises a heterogeneous group of linear binary copolymers of 1-4 linked β-D-mannuronic acid (M) and its C-5 epimer α-L-guluronic acid (G). The monomers can appear in homopolymeric blocks of consecutive G-residues (G-blocks), consecutive M-residues (M-blocks), alternating M and G-residues (MG-blocks) or randomly organized blocks.

Alginate has long been studied as a biomaterial in a wide range of physiologic and therapeutic applications. Its potential as a biocompatible implant material was first explored in 1964 in the surgical role of artificially expanding plasma volume (Murphy et al., Surgery 56:1099-108, 1964). Over the last twenty years, there has been remarkable progress in alginate cell microencapsulation for the treatment of diseases such as diabetes, amongst others.

As used herein, the term alginate-conjugates can include, but are not limited to, alginate-collagen, alginate-chitin, alginate-cellulose, alginate-laminin, alginate-elastin, alginate-fibronectin, alginate-collagen-laminin and alginate-hyaluronic acid in which the collagen, chitin, cellulose, laminin, elastin, collagen-laminin or hyaluronic acid is covalently bonded (or not bonded) to alginate.

“A,” “an,” and “the” as used herein refers to both singular and plural referents unless the context clearly dictates otherwise. By way of example, “a compartment” may refer to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

The recitation of numerical ranges by endpoints includes all numbers and fractions subsumed within that range, as well as the recited endpoints.

The expression “percentage,” “%,” or “% by weight” (weight percent), here and throughout the description unless otherwise defined, refers to the relative weight of the respective component based on the overall weight of the formulation.

In the experiments described herein, an encapsulation system was used comprising alginate that is high in mannuronic acid (high-M). Particularly good results were obtained with high-M alginate that has a low viscosity. The purity degree of the alginate has been shown to determine the biocompatibility of alginate-based particles. According to FDA requirements for device implantation, the content of endotoxin must be below 350 EU per patient (below 15 EU for CNS applications). As the physio-chemical properties of endotoxins are very similar to alginates, their removal has been a challenging task, but purified alginates with a specified endotoxin content below 100 EU/g are now commercially available. cGMP-qualification of batches requires that the alginates are characterized by validated methods according to ASTM guide 2064. A certificate should be delivered per batch.

Much of the prior research efforts have focused on the use of high-G alginates, in particular because high-G alginates provide a more robust and sturdy matrix for encapsulation than high-M alginates. This disclosure utilizes a composition comprising a high mannuronic acid alginate (High-M).

The term “high-M” is used herein to refer to alginates with a mannuronic acid content of at least 50%, preferably more than 50%. In a preferred embodiment, the mannuronic acid content is at least 55%, such as at least 60%, such as at least 65% or at least 67%, such as 68%, 70% or at least 75%. The mannuronic acid content can be obtained from naturally occurring alginates, by chemical modification, and/or by blending of different alginate batches with different mannuronic acid content.

The term “low viscosity” as used herein is meant to refer to an alginate composition with a viscosity of less than 100 mPa·s, such as less than 90, 80, 70, 60, 50, 40, 30, or less than 20 mPa·s. Particularly good results were obtained, however, when the high-M alginate had a viscosity of more than 20 mPa·s.

A skilled person is well aware of techniques to measure the viscosity of alginates. A representative example of such a method is described in ASTM F2064-00 (2006) Standard Guide for characterization and testing of alginates as starting materials intended for use in biomedical and tissue-engineered medical product application (using ASTM D2196 set at 20 degrees Celsius and 2 weight % high-M Alginate). Any other method for measuring viscosity of alginates is, however, equally well suited (the material from Pronova used herein in Example 2 may therein be used as a reference value).

In a recent paper (Writer et al., Tissue Engineering 16:1503-1513 (2010)), it was described that isolated adult porcine pancreatic islets that were encapsulated in macroscopic structures, formed from an alginate with a high mannuronic acid (High-M) content and with a high viscosity, were able to survive for a period of 12 weeks and were able to down-regulate blood glucose levels in diabetic rats for 60 days when implanted subcutaneously.

This technology suffers from the disadvantage that the macroscopic structures must be implanted by surgical methods, whereas smaller structures are preferred for implantation by injection. Unfortunately, high-M alginate with a high viscosity is inherently unsuited for the production of small, injectable particles, such as particles with a diameter between 200 and 800 micrometers.

Moreover, in the technology described by Vérner (supra), angiogenesis already decreases four weeks after implantation. That makes the method less suitable for clinical practice, since the composition must then be administered too often.

The method of Writer was repeated with low viscosity (less than 100 mPa·s) high-M (at least 50% Mannuronic acid) alginate microparticles with a diameter of between 200 and 800 micrometers. Consistent with the findings of Veriter, no angiogenesis was observed.

Surprisingly, however, when these low viscosity, high-M alginate micro-particles were loaded with aggregates of fetal, newborn or perinatal pancreatic cells with an aggregate size of less than 100 micrometers, this resulted in a long-lasting induction of angiogenesis. Moreover, cells were viable, able to reproduce, and produce insulin for a period of over 20 weeks. These results were even more apparent in cell preparations wherein more than 50% of the aggregates were below 50 micrometers.

In one aspect, the disclosure, therefore, relates to a composition for use in the treatment of a subject with diabetes mellitus wherein the treatment comprises implanting of the composition into the body of the subject at a predefined location and wherein the composition comprises aggregates of fetal, newborn or perinatal pancreatic cells encapsulated in alginate with a mannuronic acid content of at least 50%, and a viscosity of 100 mPa·s or less, wherein the aggregates are smaller than 100 micrometers.

More in particular, the disclosure relates to a composition for use as described above wherein more than 50% of the aggregates were below 50 micrometers. More than 50% in this respect incorporates values of more than 60%, such as more than 70% or even more than 80%, such as 90% or 100%. Such small aggregates are preferred because they result in an improved cell survival and cell growth (see below).

As described above, surprisingly good results were obtained when the high-M alginate had a viscosity of less than 100 mPa·s. In a preferred embodiment, the disclosure, therefore, relates to a composition for use as described above wherein the high-M alginate has a viscosity of 100 mPa·s or less, such as 90 or less than 90, such as 80, 70, 60, 50, 40, 30, or less than 20 mPa·s.

Particularly good results, however, were obtained when the high-M alginate had a viscosity of more than 20 mPa·s. In a preferred embodiment, the disclosure, therefore, relates to a use as described above wherein the high-M alginate has a viscosity of more than 20 mPa·s, such as 30, 40, 50, 60 or 70 mPa·s.

It was also found that cells, in particular VEGF-producing cells encapsulated in high-M alginate particles, are exceptionally well suited for inducing or improving angiogenesis and/or vascularization. The disclosure thus also relates to a composition for use as described above wherein the cells are VEGF-producing cells.

Also disclosed herein is a foreign body, suitable for implantation into a subject at a predefined location, wherein the foreign body comprises cells encapsulated in low viscosity, high-M alginate for use in inducing or stimulating angiogenesis.

In other terms, the disclosure relates to a method of inducing or stimulating angiogenesis, wherein a composition such as a foreign body comprising cells encapsulated in high-M alginate as described above is implanted into a subject in need of inducing or stimulating angiogenesis at a predefined location. Such a subject may be suffering from diabetes mellitus. Diabetes mellitus in this context includes type 1 or type 2 diabetes mellitus.

In yet other words, the disclosure provides a composition for use in the treatment of a subject, such as a subject with diabetes mellitus, or a subject with diabetes mellitus type 1, wherein the composition is implanted into the body of the subject at a predefined location and wherein the composition comprises aggregates of fetal, newborn or perinatal pancreatic cells encapsulated in high-M alginate, wherein more than 50% of the aggregates are below 50 micrometers. This is in contrast with various prior art publications (Dufrane et al., 2006) that describe the need for complete adult porcine islets, which implies larger diameters of aggregates (>100 micrometers).

As used herein, the term “foreign body” is meant to refer to a composition that comprises at least one component or substance that is not derived from the subject in which the foreign body is to be implanted. The term, therefore, excludes a composition consisting entirely of cells or tissue derived from the subject itself, whereas it includes a composition comprising a compound that is alien or foreign to the body of the subject in which the foreign body is to be transplanted. For example, a composition comprising high-M alginate is foreign to the body. Cells or tissue derived from the body of the subject to be transplanted is not foreign or alien to the subject.

In a preferred embodiment, the foreign body is exclusively comprised of compounds foreign or alien to the body of the subject. As an example, provided herein are foreign bodies comprising high-M alginate and porcine cells. Human cells derived from the body of a subject different from the subject in which the foreign body is to be transplanted are considered to be foreign to the subject in which the foreign body is to be transplanted, even when the subject from which the cells are derived belongs to the same species as the subject in which the foreign body is to be transplanted.

In the studies presented herein, it is shown that perinatal cells encapsulated in high-M alginate and transplanted subcutaneously were able to proliferate, differentiate and perform their normal function of responding to elevated glucose levels by producing and secreting insulin and C-peptide levels well above the minimal physiological levels starting from week 4, for at least 16 or 20 weeks, after which the experiments were terminated.

Other suitable sites for transplantation include fat depositions such as the omentum, adipose tissue, or intramuscular.

When VEGF-producing porcine beta cells were used in capsules of high-M alginate, they produced functional insulin during the entire interval of 16-20 weeks, as evidenced in the figures and tables presented herein.

Without wanting to be bound by theory, it is hypothesized that the continuous production of insulin during 16 or 20 weeks is caused by an improved blood supply to the high-M encapsulated grafts. Indeed, extensive vascularization of sub-cutaneous transplanted high-M grafts were observed, whereas high-G grafts did not show a significant pattern of vascularization (FIG. 4). High-G grafts also did not produce significant amounts of insulin during the 20 weeks follow-up period as is detailed in the examples provided herein.

It was observed that fetal, newborn or perinatal porcine cells were capable of additional in vivo growth after transplantation. This has also been recognized in the literature (M. Bogdani, K. Suenens, T. Bock, M. Pipeleers-Marichal, P. In't Veld, and D. Pipeleers: “Growth and functional maturation of beta-cells in implants of endocrine cells purified from prenatal porcine pancreas,” Diabetes 2005 December; 54(12):3387-3394). However, it was observed that this was, in particular, the case with high-M alginate encapsulated grafts, where beta cell number increased three- to six-fold in 20 weeks after transplantation. Similar growth in graft size was not present with high-G encapsulated cells where the absolute cell number decreased by 50% after 20 weeks in vivo (Table 1).

TABLE 1
In vivo changes in cellular composition and cell number between
grafts (n = 7) and explanted low viscosity, high-M capsules
(HM, n = 26) and explanted high-G capsules (HG, n = 3).

| | | Cellular | | | | |
|---|---|---|---|---|---|---|
| | Capsule | Composition (%) | | | | Beta cell |
| | recovery | Insulin | Glucagon | Cell viability | Cell recovery | recovery |
| | (%) | pos (%) | pos (%) | (%) | (fold) | (fold) |
| HM graft | NA | 40 +/− 3 | 25 +/− 5 | 87 +/− 3 | NA | NA |
| HG graft | NA | 36 +/− 4 | 28 +/− 7 | 73 | | |
| HM explant | 60 +/− 10 | 73 +/− 11 | 12 +/− 8 | 93 +/− 7 | 2.3 +/− 0.8 | 4.8 +/− 1.8 |
| HG explant | ND | ND | ND | ND | 0.5 +/− 0.15 | ND |

NA = Not Applicable,
ND = Not Determined.

The disclosure also relates to a use as described above wherein the cells are VEGF-producing cells. The amount of cells did not appear to be critical. The experiments described herein were reproduced with transplants comprising 0.5×10⁶, 1×10⁶ and 2×10⁶ cells and found identical results as obtained with transplants containing 3×10⁶ cells.

Such cells may advantageously be used in the treatment of a number of different diseases. A particularly suitable disease to be treated with the methods and compounds disclosed herein, would be diabetes mellitus. In that case, the cells would advantageously be insulin-producing cells. Such cells may be beta cells, derivable from a variety of sources. Beta cells may, for example, advantageously be derived from a neonatal or fetal pancreas. In a further preferred embodiment, the pancreas may be obtained from a porcine source. The experiments described hereinabove and in the examples were also performed with human pancreatic islet cells. The results were comparable, if not identical, with the results obtained with porcine cells. Therefore, the disclosure also relates to a use, a method, a composition or a composition for use described above wherein the cells are human cells.

It has also been shown herein that the high-M alginate grafts may advantageously be transplanted subcutaneously. This has a number of distinct advantages, first of all, of course, the ease of handling. Subcutaneous transplantation may easily be performed by injection or by other minimally invasive implantation. The foreign body may also be implanted in the subject by percutaneous placement, keyhole surgery, incisions, or catheter/tracher placement.

The term “injection” in this context is to be understood as the act or process of forcing a viscous solution in the body of a subject, thereby placing a composition in the body of the subject, by means of a syringe, optionally through a needle.

Subcutaneous transplantation is especially appealing for easy post-transplant monitoring and removal of graft if needed.

Moreover, it has been shown herein that high-M grafts transplanted subcutaneously produce increasing amounts of functional insulin during the 20 weeks follow-up period, whereas intraperitoneal grafts of high-M encapsulated cells did not produce significant amounts of insulin.

The graft or foreign body for transplantation may be in any form suitable for the intended application. When applied subcutaneously, the graft may advantageously be in the form of a gel or a particle with a largest dimension of 8000 micrometers, such as 1000 micrometers, such as 900, 800 or 700 micrometers, whereas the smallest diameter of the particles may be more than 10 such as 30, such as 50, 100, 200, or 400 micrometers. Particles may advantageously have a diameter of any value lying between the above-mentioned smallest and largest dimensions. Particularly preferred particles or capsules are essentially spherical and have a largest dimension between 10 and 8000 micrometers, such as between 30 and 1000 micrometers, such as between 50 and 900 micrometers, such as between 100 and 800 micrometers, or between 200 and 800 micrometers. The size of the particle is mainly determined by the viscosity and the flow rate of the liquid formula, and by the settings of the droplet-forming mechanism to form droplets, which is in the function of the number of cells that it is intended to harbor. A skilled person will know how to select the optimal dimensions of the capsules or particles.

The graft or foreign body may also be in the form of a mesh comprising alginate entrapped cells. It may also be in the form of a cassette (THERACYTE™) or any other biocompatible semi-permeable encapsulating device, such as a polymer scaffold.

In a further preferred embodiment, the high-M alginate has a mannuronic acid content of more than 50%.

### BRIEF DESCRIPTION OF THE DRAWINGS

FIG. 1: Graph showing blood glucose levels (mg glucose/100 ml) in mice transplanted subcutaneously with fetal porcine beta cells encapsulated in high-M alginate (solid line) versus controls (dashed line). Engrafted mice (n=15) and control, non-transplanted mice (n=5) were injected intraperitoneally with 3 gram/kg life weight glucose and their blood glucose levels were followed.

FIG. 2: Graph showing plasma porcine C-peptide levels during 20 weeks after subcutaneous transplant of porcine fetal beta cells. Solid lines are the engrafted mice; dashed lines are the non-transplanted controls.

FIG. 3: Graph showing blood glucose levels (mg glucose/100 ml blood) during 20 weeks after subcutaneous transplant of porcine fetal beta cells. Solid lines are the engrafted mice; dashed lines are the non-transplanted controls. *p<0.05; **p<0.01; ***p<0.005 student t-test, double-tailed, unpaired.

FIG. 4: Light microscopic image, showing vascularization of a high-M graft explant, 20 weeks after subcutaneous transplantation.

FIG. 5: Graph showing glucose-induced plasma porcine C-peptide (ng/ml) over a period of 20 weeks after transplantation. Solid line is a high-M graft; dashed line is a high-G graft.

FIG. 6: Graph showing fasting blood glucose levels (mg %) over a period of 20 weeks after transplantation. Solid line is a high-M graft; dashed line is a high-G graft. Asterisks indicate the level of significance between the two groups. *p<0.05; **p<0.01; ***p<0.005 student t-test, double-tailed, unpaired.

FIG. 7: Graph showing glucose-induced plasma porcine C-peptide (ng/ml) over a period of 20 weeks after transplantation. Solid line is a subcutaneous high-M graft; dashed line is an intraperitoneal high-M graft.

### DETAILED DESCRIPTION

### EXAMPLES

### Example 1

### Preparation of Cells for Encapsulation

Porcine fetal pancreatic cells were obtained essentially as previously described in EP 1146117. In particular, the following procedures were employed. Pregnant sows of 108 to 114 days of gestation were anesthetized and the fetuses removed surgically under sterile conditions in an operating theater, the fetuses (crown-rump length 28+/−5 cm (mean+/−SD)) were decapitated and the pancreases removed by dissection under aseptic conditions and collected into sterile isolation medium (Pipeleers et al., Endocrinology 117:806-816, 1985). The tissue was cut with scissors into small fragments of approximately 1 mm³ in size.

After washing the tissue fragments with isolation medium, the fragments were suspended in 200 ml isolation medium with 0.3 mg/ml collagenase-P (Roche) (room temperature) and then shaken for 15 minutes. The tissue digest was filtered through a 500-micron filter and the filtrate centrifuged through a solution with density of 1.040 (6 minutes at 1500 rpm) after which the pellet was saved. The material that was left on the 500-micron filter was again incubated with collagenase for another 15 minutes and then filtered and centrifuged as before, whereby the pellet was again saved. The material that remained on the filter the second time was resuspended in a calcium-free dissociation medium (Pipeleers and Pipeleers-Marichal, Diabetologia 20:654-663, 1981) and dispersed during a 15-minute incubation at room temperature, before filtration and centrifugation as described above; this dissociation procedure was repeated on the material that was left on the filter.

The four pellet fractions were suspended in isolation medium containing 2% newborn calf serum (NCS), and filtered through a 100-micron filter to remove large cell clusters; the filtrate was now composed of single cells and small (<100-micron diameter) cellular aggregates. This cell suspension was pumped into the chamber of a JE-10X counterflow elutriation rotor (Beckman instruments Inc., Palo Alto, Calif.) and centrifuged at 1500 rpm and a pump setting of 190 ml/minute. Under these conditions, particles with a diameter of less than 15 microns were flushed out of the chamber, which thus retained particles with a larger size (15-100 microns). The elutriated fractions were centrifuged and the pellet resuspended in a medium with a density of 1.040 and layered on top of a medium with a density of 1.075; after centrifugation for 20 minutes at 2500 rpm, the interphase between 1.040 and 1.075 was removed with a siliconized Pasteur pipette and washed with isolation medium containing 2% NCS. The cells were counted automatically in NucleoCounter YC100 Chemometec, 900-0300).

From the elutriated less than 15-micron fraction, between 30 and 50 million cells were obtained for each fetal pancreas. More than 60% of the cells contained in this fraction were single cells.

Cells were plated into 14 cm sterile bacteriological petri dishes containing HAM's F10 with 6 mM glucose, penicillin 0.075 mg/ml, streptomycin 0.1 mg/ml, 2 mM glutamine, 2 mM CaCl2, 50 uM Isobutylmethylxanthine (IBMX), 1 uM Hydrocortisone, 5 mM Nicotinamide and 0.5% Bovine Serum Albumin, with 0.5 to 1.0 million cells per ml medium. After a 16-hour culture at 37 degrees Celsius and 5% CO2 in air, the cells were washed and the medium replaced by the same type HAM's F10 as before except for a lower calcium concentration (0.2 mM). Under these conditions, the cells were kept in suspension culture for 7 days, washed with 0.9% NaCl and then harvested by centrifugation.

The cell suspension thus obtained essentially comprised cell aggregates of 100 micrometers or less. Cell aggregate fractions containing more than 50% of single cells and cell aggregates below 50 micrometers provided particularly good results in inducing angiogenesis.

### Example 2

### Encapsulation of Cells

Microencapsulation was performed using a highly purified alginate obtained from Pronova UP LVM, Norway, product number 4200206, with a high mannuronic acid content (high-M, at least 50% of M).

PRONOVA UP LVM is a low viscosity (20-200 mPa·s) sodium alginate where more than 50% of the monomer units are mannuronate. Based on the provided batch records, batches were selected and used with a viscosity of less than 100 mPa·s.

In the control experiments described herein, a high-G alginate is used. In the context of this application, high-G means a high Guluronic acid (high-G, >50% of guluronic acid or guluronate).

A 2% solution of alginate was mixed with the cell pellet obtained in Example 1, to a final concentration of 20×10⁶ cells per ml of alginate in a 50 ml Falcon tube.

The cell suspension was subsequently processed through a coaxial air flow device using the following settings:
flow rate pump: 1.8 ml/minute
air flow meter: 2.5-3 L/minute
pressure valve 1: 0.2 MPa
pressure valve 2: 0.1 MPa

Using a peristaltic pump, the cell-alginate mixture was aspirated out of the 50 ml Falcon tube using a metal hub needle (gauge 16), and advanced through a tubing toward the 22 gauge air-jet needle. Droplets containing cells in alginate were produced by extrusion (0.5-1.5 ml/minute) through a 22 gauge air-jet needle (air flow 2.5-3 l/minute).

Droplets fell 2 cm lower into a 20 ml beaker containing a gelling solution of 50 mM CaCl2 and 1 mM BaCl2 in 10 mM MOPS, 0.14 M mannitol, pH 7.2-7.4 as gelling solution. Upon contact with this buffer, the microdroplets jellified. Droplet size varied between 200-800 μm, depending on pump flow rate and on air flow used. High-M capsules were on average 625 um (+/−70) in diameter, whereas high-G capsules were 500 um (+/−75).

The droplets were left for 7 minutes in the gelling solution. Afterward, the capsules were removed from the gelling solution and gently washed with 0.9% NaCl. This step was repeated three times with each time a complete renewal of the washing solution.

After taking samples for QC, encapsulated cells were cultured in serum-free medium at 37° C. and 5% CO2 until transplantation.

### Example 3

### Quality Control of Capsules and Cell Count of Encapsulated Cells

Cell number and viability of the encapsulated cells were determined by Nucleocount (Nucleocounter YC-100, Chemometec) according to the manufacturer's instructions. In brief, 30 capsules were obtained in triplicate from the encapsulated cell suspension obtained in Example 2. Capsules were treated with Alginate Lyase from Flavobacterium multivorum obtained from Sigma Aldrich. The powder, ≧10,000 units/g solid was used according to the manufacturer's instructions.

After lyase treatment, the free cells were counted. In a typical experiment, the high-M capsules contained about 1500 cells (+/−360) per capsule, whereas the high-G capsules contained 990 cells per capsule (+/−500). Of these cells, about 40% were insulin-producing cells and about 25% were glucagon-producing cells. Between 70 and 90% of the cells appeared to be viable.

### Example 4

### Transplantation Studies

In the transplantation experiments described herein, 3×10⁶ encapsulated cells were transplanted into 8-week-old male NOD/SCID JAXMICE® (Charles River Laboratories), either intra-peritoneal (IP) or subcutaneous (SC). For IP transplantation, a small incision was made in the abdominal wall and peritoneum of the animal along the linea alba. Encapsulated cells were subsequently transferred into the peritoneal cavity using a 5 ml pipette filled with 4 ml buffer solution.

SC transplants were placed in the left dorsal subcutaneous space. For this, the skin was gently detached from the underlying muscle and grafts were transplanted in the resulting space. All surgical procedures were performed under general anesthesia.

Control animals received no implantation. The animals were then monitored for up to 20 weeks.

### Example 5

### Determining the Blood Glucose Levels

Blood glucose levels were determined in tail blood using Glucocard strips (Glucocard X-sensor; A. Menarini Diagnostics, Florence, Italy) according to the manufacturer's instructions. Glucose measurements were performed every two weeks after transplantation.

### Example 6

### Determining C-Peptide Produced by Grafts and Host

Glucose (3 gram/kg life weight) was injected intraperitoneal and porcine C-peptide was determined 15 minutes after injection in 250 μl EDTA-aprotinin plasma using a commercially available RIA kit (PCP-22K, Linco Research Inc., St-Charles, Mo., USA). This is termed herein the “glucose-induced plasma porcine C-peptide levels.”

### Example 7

### Transplants Produce Functional Insulin

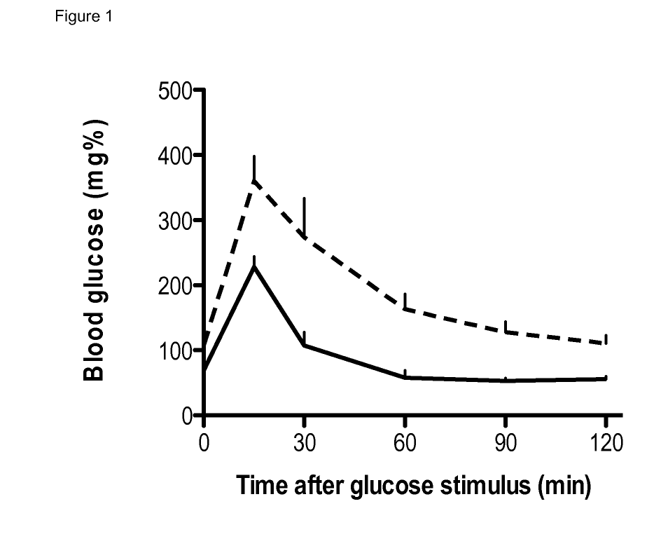

In order to prove that the transplanted grafts indeed produced functional porcine insulin, glucose levels were monitored for 2 hours after intraperitoneal injection of glucose solution. Samples were taken at 0, 15, 30, 60, 90 and 120 minutes. FIG. 1 shows that mice receiving porcine fetal beta cells encapsulated in low viscosity (<100 mPa·s) and high-M capsules subcutaneously, cleared glucose more rapidly and to a significantly lower end point than the control (non-transplanted) mice. FIG. 1 shows the results obtained with 15 transplanted mice and 5 control mice, 20 weeks after transplantation. The error bars indicate the standard deviation; results were found highly significant (p<0.001).

### Example 8

### Encapsulated Cells Survive for 20 Weeks and are Functional

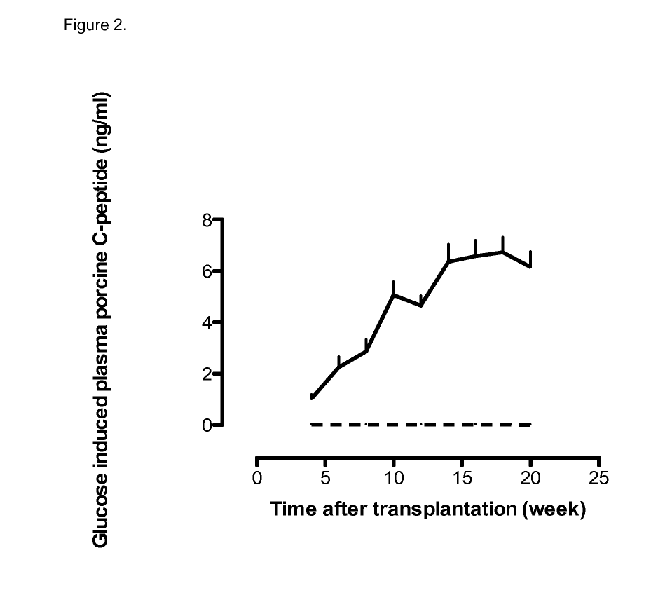

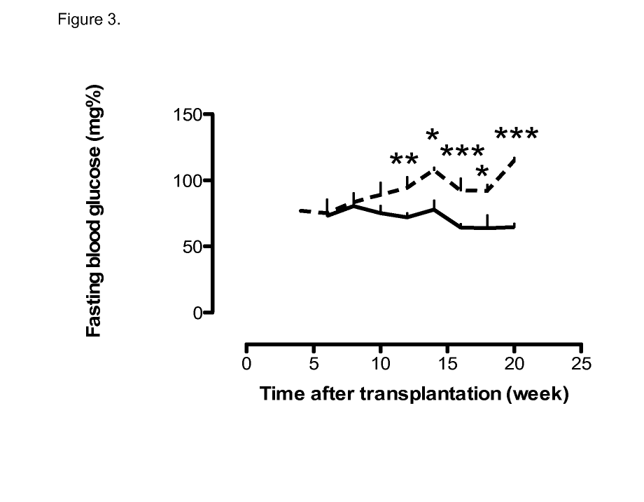

Glucose-induced plasma porcine C-peptide levels were measured every 2 weeks after transplantation. The amount of porcine C-peptide produced was taken as a measure for the production of functional insulin produced by the graft. FIG. 2 shows that mice, transplanted subcutaneously with porcine fetal beta cells in low viscosity, high-M alginate, produced an increasing level of porcine C-peptide during the entire period of 20 weeks. This reflects the production of functional insulin as evidenced by the decrease of glucose levels in blood, 20 weeks after transplant as is shown in FIG. 3.

### Example 9

### Subcutaneous Transplants in Low Viscosity, High-M Alginate Induce Angiogenesis

Animals were sacrificed at week 20 after transplantation and the grafts were removed for histopathological examination. The vascularization of the graft was determined under a light microscope.

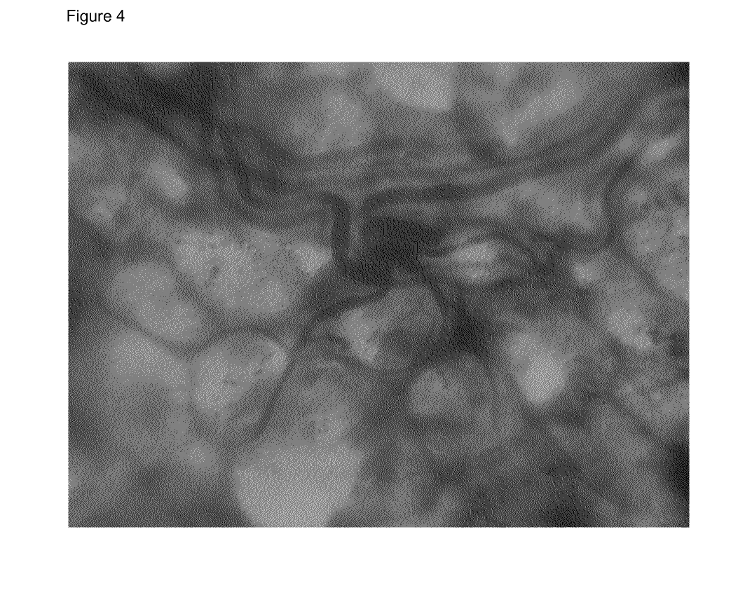

It was found that porcine fetal beta cells, encapsulated in low viscosity, high-M alginate induced angiogenesis very efficiently when implanted subcutaneously. It was found that almost every low viscosity, high-M capsule was surrounded by a capillary network (FIG. 4). This indicates a functional connection between the capillary and the graft. In contrast, the high-G capsules were not surrounded by capillaries, an occasional capillary had entered the graft, however, without making functional connections. The capillaries in the high-G grafts seemed to by-pass the capsules rather than supplying them with blood. Low viscosity, High-M capsules that were not formulated with cells also survived for 20 weeks, but did not show the remarkable capillary network, neither in Nod-Scid nor in Balb C mice (not shown).

### Example 10

### Cellular Viability After Explantation

Explants were digested with collagenase, the capsules were hand-picked and counted to determine the recovery rate. The capsules were digested with lyase as described above. Cellular viability was determined by Hoechst/propidium iodide staining. Nuclear count and viability were established using a Nucleocounter. Cellular composition was determined after immunofluorescent staining of formaldehyde-fixated cytoslides (4 minutes at 1000 rpm in Cytospin 4 (Thermo Fisher Scientific Inc.)), with guinea pig anti-insulin and rabbit anti-glucagon.

### Example 11

### Beta Cells Encapsulated in Low Viscosity, High-M Alginate Versus Cells Encapsulated in High-G

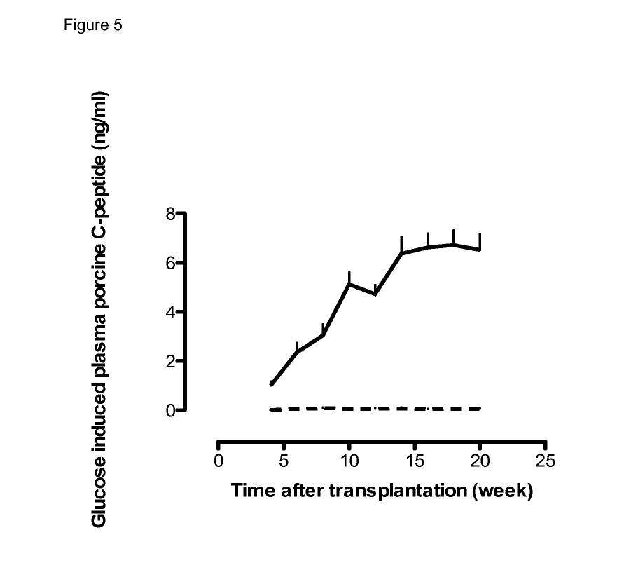

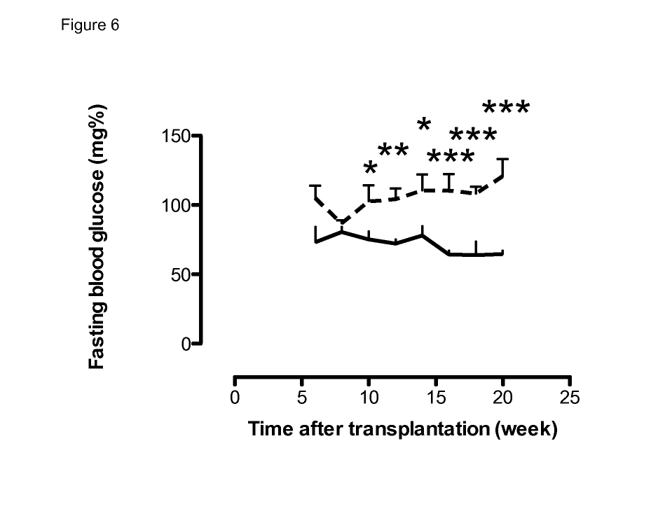

Fetal porcine Beta cells encapsulated in low viscosity, high-M alginate and transplanted subcutaneously showed a steady increase in C-peptide during the 20-week period, whereas cells transplanted in High-G particles did not produce significant levels of C-peptide. This is shown in FIG. 5. Glucose levels in animals transplanted with cells encapsulated in high-G alginate did not change significantly during the 20-week period, whereas the levels of glucose in animals receiving the low viscosity, high-M encapsulated cells significantly decreased (FIG. 6). Beta cell number increased four- to six-fold in 20 weeks after transplantation in high-M alginate grafts, whereas in high-G grafts, it was reduced by 50%.

### Example 12

### Intraperitoneal Versus Subcutaneous Transplantation

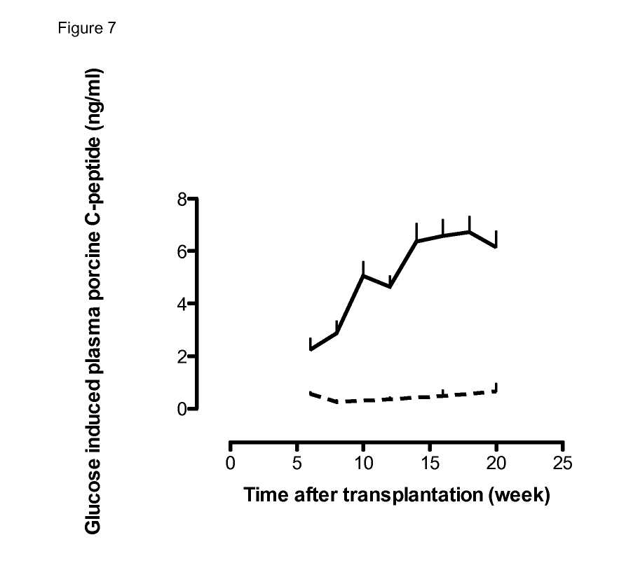

Fetal porcine beta cells encapsulated in low viscosity, high-M alginate and transplanted subcutaneously showed a significantly higher production of C-peptide during the 20-week follow-up than the same capsules when transplanted intraperitoneally (FIG. 7). As a consequence, the glucose levels of the subcutaneous transplanted animals decreased, whereas the intraperitoneal-transplanted animals showed no significant reduction of glucose levels.

### Example 13

### Comparative Analysis of Cell Preparations

The differences were determined between the cell preparations used in this study and the cells used in the prior art. Adult pancreatic islets are commonly used in prior art transplantation studies, such as the studies described in Dufrane et al., Xenotransplantation 2006: 13; 204-214. The cell preparations as used in the studies described herein are characterized by the fact that they consist of small aggregates of less than 100 micrometers, wherein more than 50%, such as 60%, 70% or even more than 80% of the aggregates are smaller than 50 micrometers. The lower limit of the aggregates (a single cell) is determined by the cell size. Good results may be obtained with aggregates larger than 15 micrometers. In contrast, Dufrane et al. (supra), described that good results were obtained with isolates made up of more than 42% of the adult islets that are much larger than 100 micrometers. Other sources (Ching et al., Arch. Surg. 136; (2001); 276-279) describe the average size of islets to be 644 micrometers.

Islets typically contain about 65% insulin-positive cells, which means cells capable of producing insulin. The cell preparation used in this disclosure contains between 30 and 40% of insulin-positive cells at the time of encapsulation. When the cell aggregates are transplanted in high-M alginate as described herein, they grow and reproduce, to such an extent that over a period of 12-20 weeks, up to 80% of the cell mass inside the high-M particles are insulin-positive cells. Other cell types present at the time of encapsulation were Glucagon-positive cells (27%+/−4), Somatostatin-positive cells (16%+/−2) Pancreatic polypeptide-positive cells (5%) and CK7 cells (15%+/−14).

Most importantly, an increase in the insulin-producing cell mass was found after transplantation of a factor 3-6, whereas the total cell mass increased by a factor 1.5 to 2.5. No increase in insulin-positive cells has ever been observed in transplanted islets; on the contrary, cell mass is totally lost after several weeks.

The cell preparation disclosed herein also appeared to include a pool of progenitor cells that differentiate and mature into insulin-producing cells, which may also contribute to an increase in cell mass. Islets are known to contain virtually no progenitor cell activity in vivo.

It was also observed that the cell aggregates as described herein had an optimal insulin secretion starting from week 4, whereas isolated islets produced insulin from the start of transplantation (Veriter et al., supra; and Dufrane et al., supra). Moreover, the immediate response of islets lasted for only four weeks; whereas the cell aggregates disclosed herein produced insulin up to 20 weeks after transplant, without evidence of reducing production and responsiveness.

1. A method of treating a subject with diabetes mellitus, the method comprising:
implanting a composition into the body of the subject at a predefined location, wherein the composition comprises:
aggregates of fetal, newborn, or perinatal pancreatic cells encapsulated in alginate particles with a mannuronic acid content of at least 50%, and a viscosity of 100 mPa·s or less, and wherein the aggregates are smaller than 100 micrometers.

2. The method according to claim 1, wherein more than 50% of the aggregates are below 50 micrometers.

3. The method according to claim 1, wherein the alginate has a viscosity of more than 20 mPa·s.

4. The method according to claim 1, wherein the cells are VEGF-producing cells.

5. The method according to claim 1, wherein the cells are insulin-producing cells.

6. The method according to claim 1, wherein the cells are beta cells.

7. The method according to claim 1, wherein the cells are of porcine origin.

8. The method according to claim 1, wherein the subject is a human.

9. The method according to claim 1, wherein the predefined location is subcutaneous.

10. The method according to claim 1, wherein the diabetes mellitus is type 1 diabetes mellitus.

11. The method according to claim 1, wherein the particles have a dimension between 100 and 8000 micrometers.

12. The method according to claim 1, wherein the particles have an average dimension between 200 and 800 micrometers.

13. The method according to claim 1, wherein the implantation is performed by injection or surgical implantation.

14. The method according to claim 1, wherein the alginate comprises at least 60% or 65% or more of mannuronic acid.
